(19) United States
(12) Patent Application Publication
(10) Pub. No.: US 2017/0113016 A1
(43) Pub. Date: Apr. 27, 2017

(54) BABY BLANKET

(71) Applicant: Serge V. Monros, Costa Mesa, CA (US)

(72) Inventor: Serge V. Monros, Costa Mesa, CA (US)

(21) Appl. No.: 15/226,580

(22) Filed: Aug. 2, 2016

Related U.S. Application Data
(60) Provisional application No. 62/245,466, filed on Oct. 23, 2015.

(51) Int. Cl. A61M 21/02; A47G 9/02

(52) U.S. Cl. CPC A61M 21/02; A47G 9/0223; A47G 2009/006

(57) ABSTRACT

An infant blanket provides soothing sounds and music so as to comfort and make quiet an infant. Within a head or face portion of the blanket, a pocket is provided into which a speaker is inserted. The speaker may connect by wire or wirelessly to a playback device. The speaker is padded by a foam pillow so as to not be noticed by an infant lying on the blanket. The playback device may have pre-programmed songs or sounds, but may also be programmable by the user. The blanket may be in the shape of a friendly animal.

### RELATED APPLICATION

This application claims the benefit of U.S. Provisional application No. 62/245,466, filed on Oct. 23, 2015.

### FIELD OF THE INVENTION

The present invention generally relates to baby blankets. More specifically, the present invention relates to a baby blanket that soothes the infant by playing sounds and music that may be uploaded on a playback device connected to a speaker in the blanket.

### BACKGROUND OF THE INVENTION

Small blankets have frequently been used to provide warmth and comfort for infants during the first few years of life. In order to provide the infant with a more enjoyable blanket, improvements in infant blankets have arisen over the years. Generally, familiar sounds experienced by the infant prior to birth, such as a heartbeat, tend to soothe and quiet the infant so that both it and its parents may relax. Soft music similarly may soothe and quiet an infant.

Several attempts have been made previously in order to provide infants or young children with a soothing environment. Some attempts have been made in the following listed art for which brief descriptions are given.

B. J. Fornarelli. U.S. Pat. No. 5,205,811. This patent is directed to a baby blanket with a heartbeat simulator. The infant blanket is shown in FIG. 1 and is formed from a padded material 12 with a pouch 13 with access by means of zipper 20. FIG. 2 shows the device that has been opened to receive heart simulator 18. The simulator is cased within a foam form, which is placed into the blanket recess and is activated when the baby is placed into the area overlying the pouch portion.

Kamirin-Balfour. U.S. Pat. No. 7,346,949. This patent is directed to an infant blanket in the shape of a bear. The blanket consists of two removable and adjustable barriers that cradle the infant. One of these barriers contains a playback unit with memory that includes multiple stored sounds, including a rhythmic heartbeat and one or more musical songs or lullabies.

Coviello. U.S. Application Publication No. 2002/012076A1. This patent application is directed to an infant blanket that contains a recording and playback device. The playback device is small and flat so as to be readily carried unobtrusively within the blanket. The device may be used to record a heartbeat, which may then be played back for an extended period of time to soothe and to help an infant to fall asleep.

S. V. Monros. U.S. Application Publication No. 2014/0066692AW. This patent application is directed to an infant blanket that uses a hydrophone to transmit heartbeat-like sounds to an infant through a fluid-filled sac, or bladder. The hydrophone is connected to a digital playback device containing a digital recording of the heartbeat. When switched on, the digital playback unit continuously repeats the heartbeat sound for the benefit of the infant.

Despite the developments in previous devices related to baby blankets, no previous baby blankets are known that contain a playback device that may have new sounds and songs uploaded to it. Furthermore, no previous baby blankets are known that contain a speaker which wirelessly connects to a playback device outside of the blanket.

Accordingly there is a need for a baby blanket which provides a variety of sounds and songs by providing a playback device that may have new sounds and songs uploaded to it. Furthermore, there is a need for a baby blanket that contains a speaker that wirelessly connects to a playback device. The present invention fulfills these needs and provides other related advantages.

### SUMMARY OF THE INVENTION

The present invention is embodied in a baby blanket having an outer shell, a layer of batting, a pillow, a speaker, a plurality of SD cards, and a playback device. An infant may be placed on the baby blanket with the infant's head resting on the pillow where the infant will hear soothing music and sounds produced by the speaker.

The outer shell has a front side and a back side which define an inner pocket within the outer shell. The outer shell may be designed to resemble a friendly animal such that the outer shell is comprised of a head, a face, ears, a torso, arms, legs, and a tail. For example, the outer shell may resemble a cow further comprising horns and hooves. The outer shell may also be designed to resemble a dog further comprising paw pads. The outer shell has a closeable opening disposed through the back side of the outer shell so as to allow for access to the inner pocket.

The layer of batting is uniformly disposed within the inner pocket of the outer shell. The batting provides padding to enhance the comfort of the blanket for an infant placed thereon.

The pillow is also disposed within the inner pocket of the outer shell. Preferably, the pillow is disposed within the head portion of the outer shell. The pillow pads and masks the presence of the speaker which is disposed beneath the pillow.

The SD cards are preprogrammed with a variety of soothing music and sounds. The playback device is capable of reading the SD cards through an SD card slot. The playback device is operably connected to the speaker, preferably through a wireless connection. The playback device has selection buttons used to switch between the songs or sounds on the SD card being read. The playback device also has volume buttons to increase or decrease the volume of the speaker.

Although an embodiment has been described in detail for purposes of illustration, various modifications may be made without departing from the scope and spirit of the invention. Accordingly, the invention is not to be limited, except as by the appended claims.

### BRIEF DESCRIPTION OF THE DRAWINGS

The accompanying drawings illustrate the invention. In such drawings:

FIG. 1 is a front plan view of a baby blanket embodying the present invention in the shape of a cow;

FIG. 2 is a back plan view of the baby blanket of FIG. 1;

FIG. 3 is a front plan view of a baby blanket similar to FIG. 1 in the shape of a dog;

FIG. 4 is a cut-away fragmented perspective view of the head portion of the baby blanket of FIG. 1 illustrating a speaker located inside the blanket; and

FIG. 5 is an environmental view illustrating the speaker of the baby blanket being controlled by an MP3 player device.

### DETAILED DESCRIPTION OF THE PREFERRED EMBODIMENTS

As shown in the drawings and for purposes of illustration, the present invention is directed to a baby blanket generally referred to by the reference number 10. The baby blanket 10 is designed to comfort, sooth, and quiet an infant using the blanket 10. The blanket 10 may generally be comprised of a soft and warm material housing a speaker that is connected to an MP3 player. The MP3 player may contain preprogrammed sounds or songs, but is also programmable by the user.

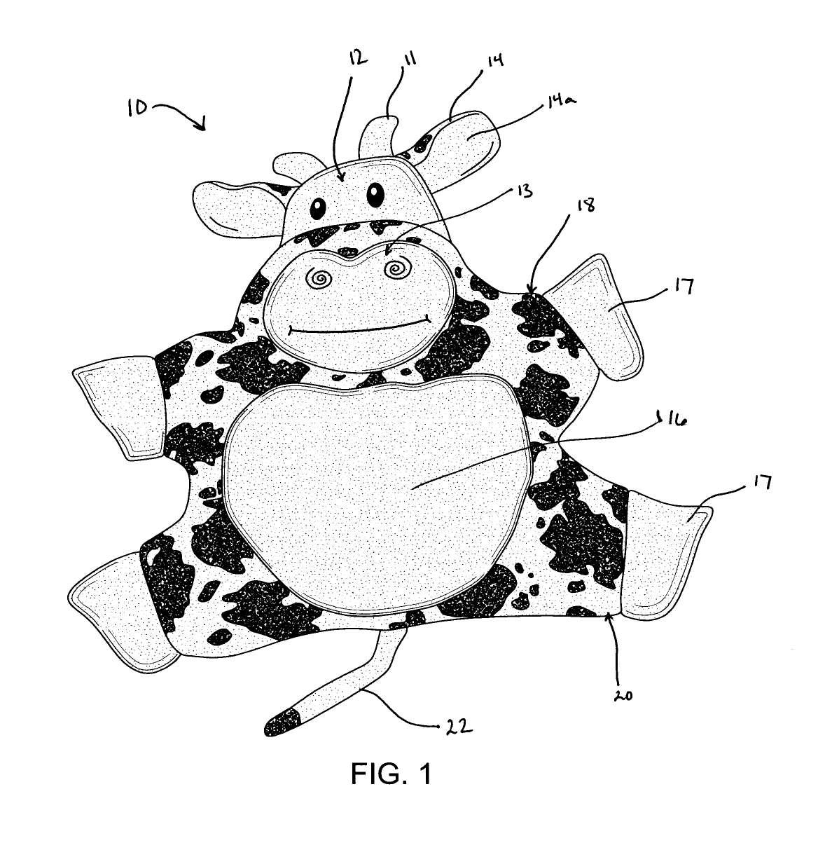

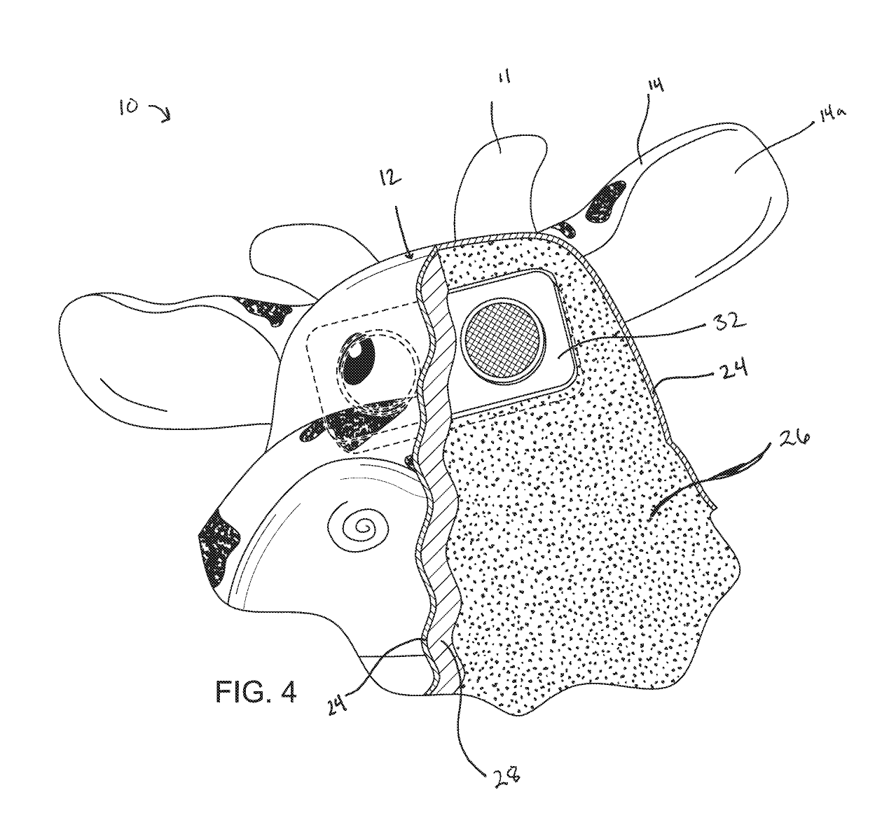

FIG. 1 is a top plan view of the baby blanket 10 illustrating that the baby blanket 10 may be designed to be in the shape of a friendly animal generally having a head 12, face 13, ears 14, a torso 16, arms 18, legs 20, and a tail 22. The baby blanket 10 may be comprised of an outer shell 24, batting 26, a pillow 28, and a speaker 32 as best illustrated in FIG. 4.

More specifically, FIG. 1 illustrates a cow embodiment of the baby blanket 10. The cow embodiment of the baby blanket 10 has horns 11 on top of the head 12 and hoofs 17 on the ends of the arms 18 and legs 20. The torso 18, hoofs 17, and inner ears 14a may be the same color and also may be made from a different material than the rest of the baby blanket 10 for design purposes. A large portion of the outer shell 24 may be white with black spots, as illustrated, to resemble a dairy cow.

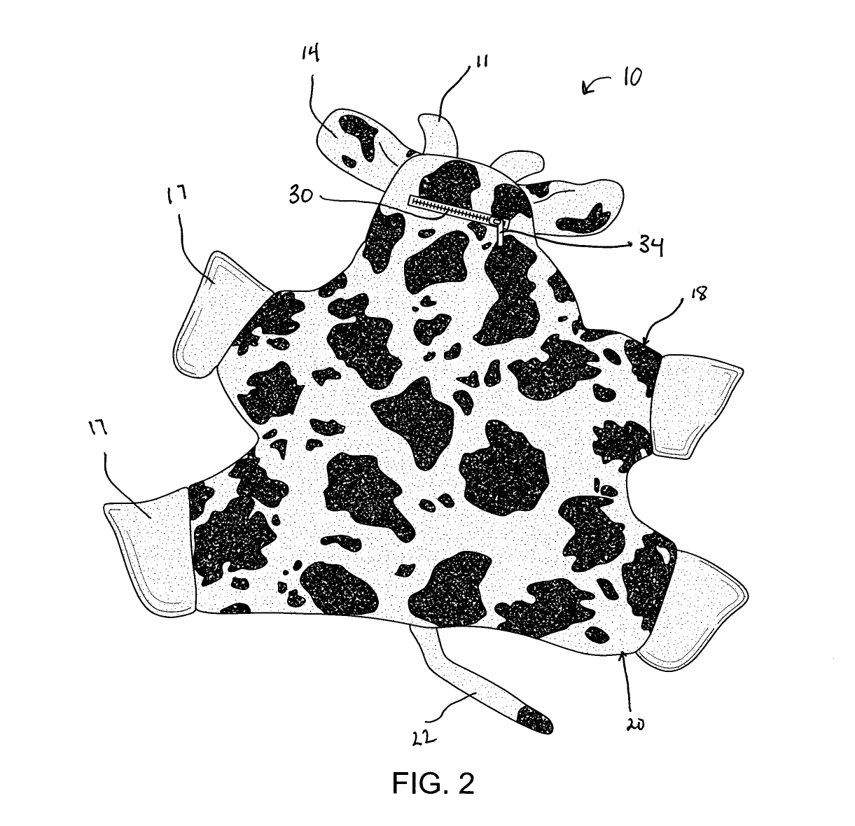

FIG. 2 illustrates the backside of the baby blanket 10. An opening 30 is located at the head portion 12 of the backside of the baby blanket 10. The opening 30 allows access to a speaker 32 (shown in FIG. 4) that is contained in the head 12 of the baby blanket 10. The opening 30 may preferably be closed by a zipper 34, but may alternatively be closed by a button, snaps, Velcro, etc.

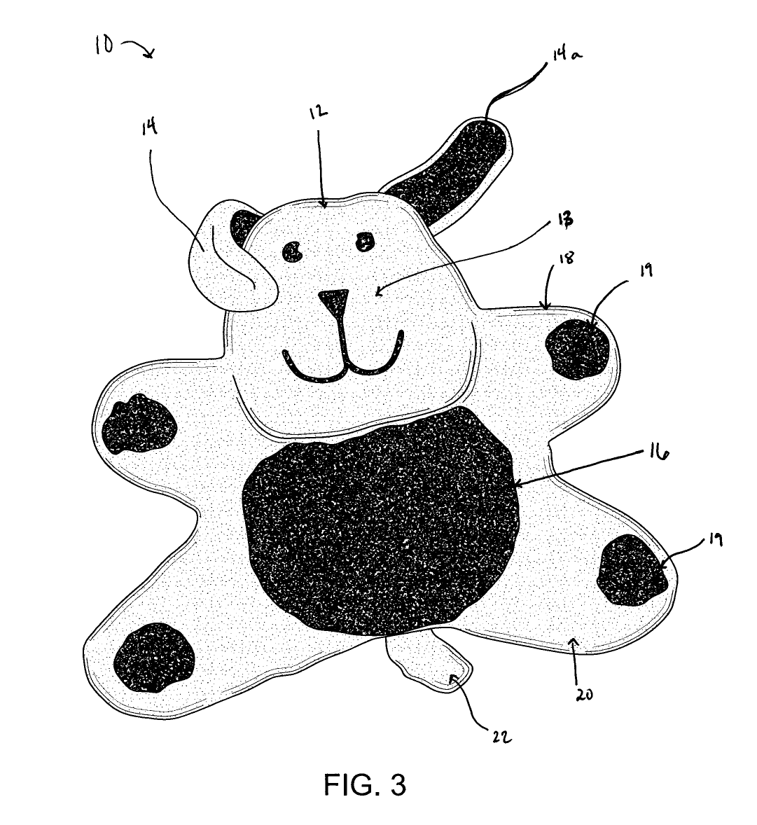

FIG. 3 illustrates an alternate embodiment of the baby blanket 10 resembling a dog. This dog embodiment similarly has a head 12, face 13, ears 14, a torso 16, arms 18, legs 20, and a tail 22. The dog baby blanket 10 also has paw pads 19 on the ends of the arms 18 and legs 20. Similar to the cow embodiment, the paw pads 19, torso 16 and inner ears 14a may be of a different color and material from the rest of the baby blanket 10 as illustrated. The figure is meant to further illustrate that the baby blanket 10 is not limited to a cow embodiment, but may resemble a dog, a cat, a bear, or any other friendly animal.

FIG. 4 illustrates a cut-away fragmented portion of the head 12 of the baby blanket 10. The figure shows that a speaker 32 may be located at the superior portion of the head 12 of the baby blanket 10. The speaker preferably is capable of connecting wirelessly to an external play back device such as an MP3 player 36. The speaker 32 may softly emit comforting music or sounds to comfort and quiet an infant using the baby blanket 10.

FIG. 4 further illustrates the general make up of the baby blanket 10. The baby blanket 10 may be composed of an outer shell 24 designed to resemble a friendly animal. The outer shell 24 may be made of cotton, fleece, cashmere, satin, or other material that is warm and soft. The outer shell 24 may be composed completely of one material or may be composed of a combination of different materials. For example, the torso 16 and parts of the head 12 may be made of satin, while the rest of the baby blanket 10 is made of a soft cotton material. A layer of batting 26 is enclosed within the outer shell 24 to provide padding for an infant lying on top of the baby blanket 10. The batting 26 may be made of cotton, polyester, wool, or other similarly padded material. A pillow 28 is also enclosed within the outer shell 24 on top of the batting 26, and is preferably confined to the head 12 of the baby blanket 10. The pillow 28 provides a cushion to support an infant's head and also to prevent discomfort by padding and masking the presence of the speaker 32. The pillow 28 may be made of foam, polyester, or other material that will support and provide a cushion for an infant's head.

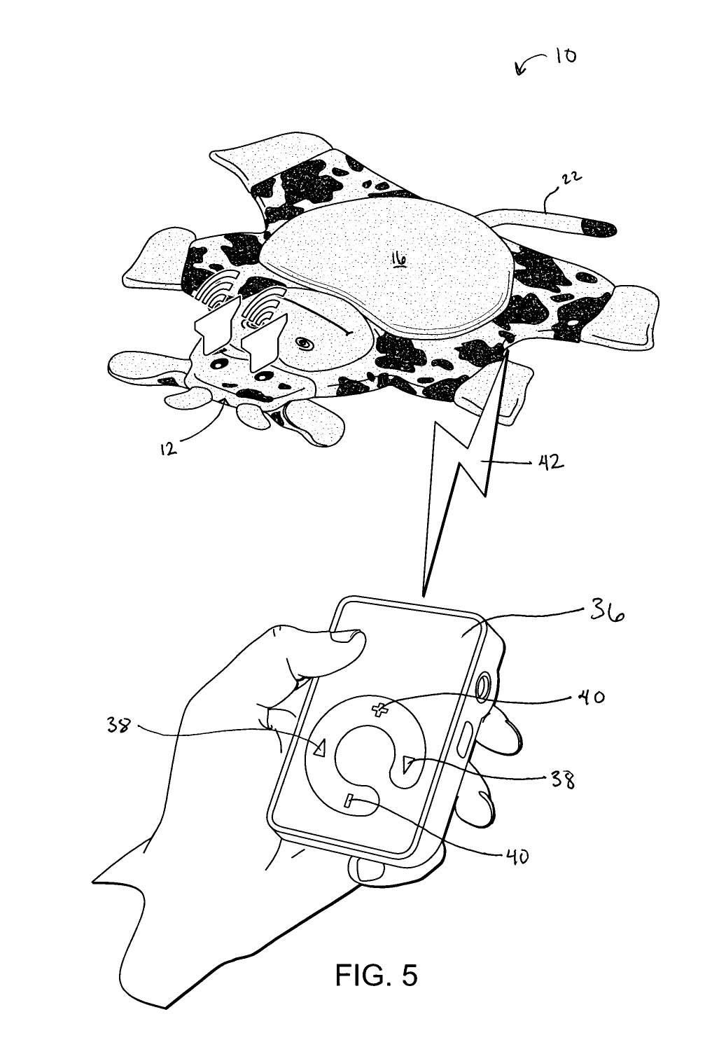

FIG. 5 is an environmental view of the baby blanket 10 illustrating how the speaker 32 is connected to and controlled by the MP3 player 36. The speaker 32 preferably connects to the MP3 player 36 though a wireless connection 42, but may also connect to the MP3 player 36 by a wire. The MP3 player 36 has selection buttons 38 to select the desired song or sound. The MP3 player 36 also has volume control buttons 40 to allow the user to adjust the volume of the speaker 32. The maximum volume should be loud enough to be audible to one pressing their ear to the face 13 of the baby blanket 10 and preferably a little louder so that one's ear adjacent to the head 12 of the blanket 10 will notice the sound. The volume range of the speaker 32 is limited in this manner so as to not cause damage to an infant's ears. The MP3 player 36 preferably has an SD card slot in which pre-programmed or programmable SD cards may be inserted. The baby blanket 10 may include pre-programmed SD cards with royalty free or royalty tunes in every genre including religions, sounds, educational material, etc. The user may also program blank SD cards with desired sounds or music for use in the MP3 player 36. The MP3 player 36 may also contain lithium batteries that may be charged through connection to a power outlet or computer by means of a USB cable.

To use the baby blanket 10, a parent or guardian may place the blanket 10 on a flat surface or floor as illustrated. An infant may then be placed on the blanket with the head of the infant on the head 12 of the baby blanket 10. The parent or guardian may then select which song or sound they would like to be played by the controls on the MP3 player 36. As the infant becomes comfortable with the environment, the infant will notice the songs and sounds coming from the baby blanket 10 and be comforted and lulled to sleep.

Although several embodiments have been described in detail for purposes of illustration, various modifications may be made without departing from the scope and spirit of the invention.

What is claimed is:

1. A baby blanket for comforting an infant, comprising:
an outer shell having a front side and a back side defining an inner pocket within the outer shell;
a layer of batting uniformly disposed within the inner pocket of the outer shell;
a pillow disposed within the inner pocket of the outer shell;
a speaker disposed within the inner pocket of the outer shell; and
a playback device operably connected to the speaker, wherein the playback device is capable of reading secure digital (SD) cards.

2. The baby blanket of claim 1, further comprising a closeable opening disposed through the backside of the outer shell so as to allow for access to the inner pocket.

3. The baby blanket of claim 1, further comprising a plurality of SD cards, wherein the SD cards are preprogrammed with soothing music and sounds.

4. The baby blanket of claim 3, wherein a portion of the plurality of SD cards are blank so as to be programmable by a user.

5. The baby blanket of claim 1, wherein the outer shell is designed to resemble a friendly animal.

6. The baby blanket of claim 1, wherein the pillow pads and masks the presence of the speaker.

7. The baby blanket of claim 1, wherein the speaker has a maximum volume just loud enough to be audible to an infant resting its head on the pillow.

8. The baby blanket of claim 1, wherein the playback device is wirelessly connected to the speaker.

9. The baby blanket of claim 1, wherein the playback device comprises selection buttons and volume buttons, wherein the selection buttons are used to switch between the songs or sounds on the SD card being read and the volume buttons increase or decrease the volume of the speaker.

10. A baby blanket for comforting an infant comprising:
an outer shell having a front side and a back side defining a pocket within the outer shell; wherein the outer shell is designed to resemble a friendly animal;
a layer of batting uniformly disposed within the pocket of the outer shell;
a closeable opening disposed through the back side of the outer shell so as to allow for access to the inner pocket;
a pillow disposed within the inner pocket of the outer shell;
a speaker disposed beneath the pillow such that the pillow pads and masks the presence of the speaker, wherein the speaker has a maximum volume just loud enough to be audible to an infant resting its head on the pillow; and
a playback device operably connected to the speaker, wherein the playback device is capable of reading SD cards.

11. The baby blanket of claim 10 further comprising a plurality of SD cards, wherein the SD cards are preprogrammed with soothing music and sounds.

12. The baby blanket of claim 11, wherein a portion of the plurality of SD cards are blank so as to be programmable by a user.

13. The baby blanket of claim 12, wherein the playback device is wirelessly connected to the speaker.

14. The baby blanket of claim 13, wherein the playback device comprises selection buttons and volume buttons, wherein the selection buttons are used to switch between the songs or sounds on the SD card being read and the volume buttons increase or decrease the volume of the speaker.
